(19) United States
(12) Patent Application Publication
(10) Pub. No.: US 2025/0391539 A1
(43) Pub. Date: Dec. 25, 2025

(54) A method and system for personalized nutrition management with food image recognition models using deep learning

(71) Applicant: CHONGQING UNIVERSITY OF POSTS AND TELECOMMUNICATIONS, Chongqing (CN)

(72) Inventors: HaiYan YU, Chongqing (CN); RenYing XU, Chongqing (CN); Jiang YU, Chongqing (CN); Shan ZHU, Chongqing (CN); ChengXin TANG, Chongqing (CN); XingYu SU, Chongqing (CN); ShengXiang ZHANG, Chongqing (CN)

(21) Appl. No.: 18/035,516

(22) PCT Filed: Sep. 5, 2022
(86) PCT No.: PCT/CN2022/117032

§ 371 Date: Aug. 25, 2024

(30) Foreign Application Priority Data
Feb. 25, 2022 (CN) 202210180116.2

(51) Int. Cl. G16H 20/60; G06V 10/26; G06V 10/42; G06V 10/44; G06V 10/764; G06V 10/77; G06V 10/774; G06V 10/776; G06V 10/82; G06V 20/68; G06V 20/70

(52) U.S. Cl. CPC G16H 20/60; G06V 10/273; G06V 10/764; G06V 10/7715; G06V 10/7747; G06V 10/776; G06V 10/82; G06V 20/68; G06V 20/70; G06V 10/42; G06V 10/44

(57) ABSTRACT

The present invention includes techniques for food image processing and particularly relates to a method and system for personalized nutrition management with food image recognition models using deep learning. The method comprises: a user side obtains an food image to be taken by a user, and the food image is input into a trained food image recognition models using deep learning to obtain different types of food sub-images; computing the amount of nutrients contained in the food sub-images, and accumulating the nutrients in all the food to obtain the total nutrients intake of the user; setting intake thresholds of various nutrients, and comparing the total intake of various nutrients with corresponding nutrient intake thresholds to obtain a comparison result; according to the comparison result, type and quantity of taken food are adjusted, and nutrition management is completed. The invention associates the food intake information uploaded by the user with other data sets (e.g., recommendations from their nutritional physician) through the server to determine whether the obtained energy and nutrient ratio are appropriate, and finally, the analyzed data is feedbacked to the user, thereby prompting the user to improve the diet plan.

### TECHNICAL FIELD

The invention includes techniques for food image processing and relates to a method and system for personalized nutrition management with food image recognition models using deep learning.

### BACKGROUND

With the improvement in living quality, people pay more attention to their own health which is closely related to the food that the human body takes every day. Therefore, the rationality of the daily diet plays an important role in the health of the body, and the key to judging the rationality of the diet plan is the identification of the type of food intake and the accurate estimation of their amount. Common dietary intake information acquisition tools include a weighing method, dietary reviews and food frequency questionnaires (FFQ). The weighing method requires weighing each food before and after meals, so as to obtain information on the type and amount of the consumed food. Although this method is accurate, it is time-consuming, laborious, and inoperable, which is only suitable for small-sample investigation. Meal review relies on the subject to recall all the food types and portions consumed in a short period of time in the past, but the review time of this method should not be too long (usually 24 hours or 72 hours); otherwise, it is easy to forget. This method reflects short-term dietary intake, but cannot reflect long-term dietary intake; FFQ can be used in large samples and can reflect the dose-dependent relationship between food types, intake and disease over a long period of time. However, the accuracy of FFQ also depends on the memory and the education level of the patients, and the error of FFQ evaluation of dietary intake can be as high as 50%. Therefore, there is an urgent need for a nutrition management method that can not only reflect the nutrition information of the user's intake for a long time, but also efficiently and accurately evaluate the dietary intake.

### INVENTION CONTENT

The present invention provides a method and system for personalized nutrition management with food image recognition models using deep learning, which comprises: a user side obtains an food image to be taken by a user, and the food image is input into a trained food image recognition models using deep learning to obtain different types of food sub-images; computing the amount of nutrients contained in the food sub-images, and accumulating the nutrients in all the food to obtain the total nutrients intake of the user; setting intake thresholds of various nutrients, and comparing the total intake of various nutrients with corresponding nutrient intake thresholds to obtain a comparison result; according to the comparison result, type and quantity of taken food are adjusted, and nutrition management is completed.

Preferably, the process of training the food image recognition models using deep learning comprises:
Step 1: obtaining a food image dataset which contains different food images;
Step 2: pre-processing the data in the food image dataset, and dividing the pre-processed food images to obtain a training set and a testing set;
Step 3: using the object region detection algorithm to segment the food images in the training set into individual masks;
Step 4: performing feature extraction on each mask, obtaining global features and local features of each mask, and performing individual feature channel classification on each feature;
Step 5: using a new decision-making algorithm for tensor feature fusion to merge the global features and local features to obtain the target frame;
Step 6: segmenting the food image according to the target frame; separating the pixel areas of different categories and different foods in the food image to complete general segmentation of the food image;
Step 7: identifying whether the types of each food image are the same, if yes, classifying semantic of each area and mark the category of each food image; if not, taking the food image as a new input, and returning to Step 4;
Step 8: numbering each food image based on semantic segmentation, outputting the image set.

Further, the pre-processing of the data in the food image dataset includes deduplication, image completion and image enhancement.

Further, the process of segmenting the food images in the training set into individual masks by the object region detection algorithm includes:
Step 1: binarizing the food image to obtain a binarized image; extracting 3 channel values or 1 channel value of each pixel in the binarized image;
Step 2: extracting contour type of the food image, and saving contour information by using approximation method; each element in the contour information saves a set of point set vectors composed of continuous food image points, and each set of food image point set represents a contour which is used as a feature for food image classification;
Step 3: segmenting the food image according to the contour information; after segmenting, returned image is the mask.

Further, the process of performing individual feature channel classification to the global features and local features of each mask comprises:
Step 1: performing affine transformation and feature extraction on the global information of each food image to obtain global features;
Step 2: performing feature extraction from each area in the food image, and fusing the local features of each area; the feature extraction methods include slices, segmented food information, and grids;
Step 3: using a deep learning network to classify individual feature channels.

Further, the decision-making algorithm of tensor feature fusion is used to merge the global features and local features, which includes:
Step 1: pre-processing the food image data, which includes subtracting the feature mean from each feature value, so that each feature has the same zero mean and variance; using tensors to construct the data structure of the 3 channels of the food image;
Step 2: computing covariance matrix of the tensor data, finding eigenvalues of the covariance matrix, arranging them from large to small, and selecting first k eigenvalues as number of features after dimensionality reduction;
Step 3: extracting the eigenvectors corresponding to the first k eigenvalues of the tensor data, so as to convert high-dimensional feature tensor into a k-dimensional eigenvector, and the k-dimensional eigenvector is the feature vector after dimensionality reduction fusion.

A system for personalized nutrition management with food image recognition models using deep learning, wherein the system includes: user side, cloud service provider and server;
the user side is used to acquire the food image to be taken by the user, and the food image is sent to the cloud service provider;
the cloud service provider is used to process the food images to obtain the user's total intake of various nutrients; the cloud processes the food images, which includes inputting the food images into the food image recognition models using deep learning to obtain different types of food sub-images; computing the amount of nutrients contained in different types of food sub-images, and accumulating the nutrients in all foods to obtain the user's total intake of various nutrients;
the server is used to obtain the user's total intake of various nutrients, compare the user's total intake of various nutrients with the intake thresholds of various nutrients, generate a diet adjustment plan according to the results, and then send the plan to the user side.

To achieve this objective, the present invention provides a computer-readable storage medium which stores computer program, wherein the computer program is executed by a processor to realize the method and system for personalized nutrition management with food image recognition models using deep learning.

The present invention provides a device for personalized nutrition management with food image recognition models using deep learning, wherein it includes a processor and a memory; the memory is used to store computer programs; the processor is connected to the memory for executing the computer program, so that the device executes the method for personalized nutrition management with food image recognition models using deep learning.

The benefits of the present invention are that the system can associate the food intake information uploaded by the user with other data sets (e.g., recommendations from their nutritional physician) through the server to determine whether the obtained energy and nutrient ratio are appropriate, and finally, the analyzed data is feedbacked to the user, thereby prompting the user to improve the diet plan. The application of the system enables monitoring of daily dietary intake in the elderly population in a follow-up cohort of nutrition and chronic diseases and further help clinical cohort studies.

Other advantages, objectives and features of the present invention will be illustrated in the following description and will be apparent to those skilled in the art based on the following investigation or can be taught from the practice of the present invention.

### DESCRIPTION OF DRAWINGS

To enable the purpose, the technical solution and the advantages of the present invention to be more clear, the present invention will be preferably described in detail below in combination with the drawings, wherein:

FIG. 1 is a schematic diagram of the method for personalized nutrition management with food image recognition models using deep learning;

FIG. 2 is a flowchart of the image segmentation;

FIG. 3 is a diagram of the food image segmentation recognition results;

FIG. 4 is an encoding schematic diagram of the food image segmentation system;

FIG. 5 is a flowchart of the food image classification;

FIG. 6 is a flowchart of the image recognition system.

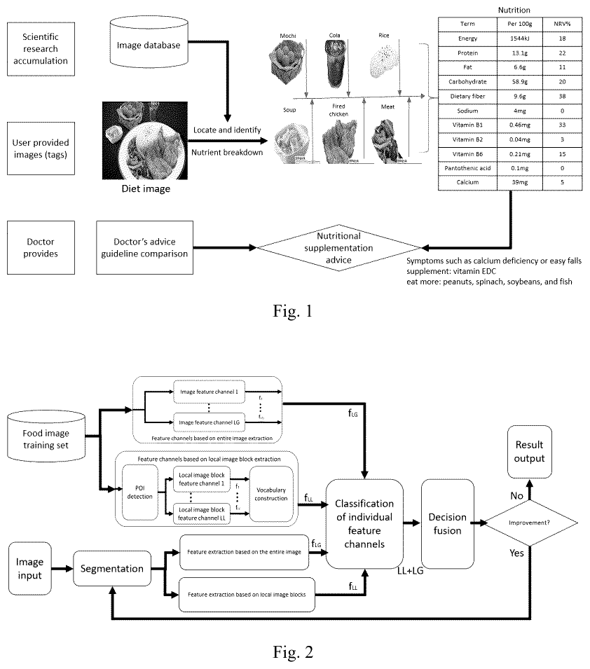

### DETAILED DESCRIPTION

Embodiments of the present invention are described as follows. Those skilled in the art can understand the related advantages and effects of the present invention through the disclosure of the description. The present invention can also be implemented or applied with additional specific embodiments. All details in the description can be modified or adapted based on different perspectives and applications without departing from the essential content of the present invention. It should be noted that the figures provided in the following embodiments only exemplarily explain the basic conception of the present invention, and if there is no conflict, the following embodiments and their features can be mutually combined.

A method for personalized nutrition management with food image recognition models using deep learning, wherein the method comprises: a user side obtains an food image to be taken by a user, and the food image is input into a trained food image recognition models using deep learning to obtain different types of food sub-images; computing the amount of nutrients contained in the food sub-images, and accumulating the nutrients in all the food to obtain the total nutrient intake of the user; setting intake thresholds of various nutrients, and comparing the total intake of various nutrients with corresponding nutrient intake thresholds to obtain a comparison result; according to the comparison result, type and quantity of taken food are adjusted, and nutrition management is completed.

A specific embodiment of the method for personalized nutrition management with food image recognition models using deep learning is that the method includes segmenting food images, analyzing nutritional components, comparing guidelines based on doctor's advice, and recommending diet plans. In the process of nutrient component identification, the ratio (p %) of each nutrient and the total weight (m) of food taken by the user are obtained from the image, and then the total amount (mp %) of various ingredients can be computed. The diet plan is based on the doctor's advice guidelines and the total composition of ideal intake which are compared to obtain the recommended food intake. Specific steps include:
Step S11: classifying the food and computing the amount of nutrients contained in each type of food according to the diet images provided by the user;
Step S12: accumulating the nutrients in all foods to compute the total intake of various nutrients in 24 hours;
Step S13: comparing the computation results with the levels of people of the same age, sex and labor intensity in the “Resident Dietary Nutrient Reference Intake” to evaluate the nutrient intake level and give nutritional recommendations;
the formula for computing the total intake of various nutrients is:

$$\frac{X}{RNI} \times 100 \% = NRV$$

wherein X is the content of a certain nutrient in 100 g of food; RNI is the recommended food nutrient intake. NRV means the ratio of the nutrient component in 100 g of food to the daily intake of the nutrient. There is a theoretical error tolerance range in this comparison that the nutritional component exceeds or is less than a certain range, which is considered to be a reasonable diet plan.
Step S14: in the process of nutrient component identification, the ratio p % of each nutrient and the total weight m of food taken by the user are obtained from the image, and then the total amount (mp %) of various ingredients can be computed. The diet recommendation is based on the doctor's advice guidelines and the total composition of ideal intake which are compared to obtain the recommended food intake max{(q−p), 0};
Step S15: the diet recommendation is based on the comparison of the total amount computed by the image recognition system with the doctor's advice guidelines (q %) to obtain the recommended food intake max {(q−p), 0} and provide a dietary assessment report using deep learning.

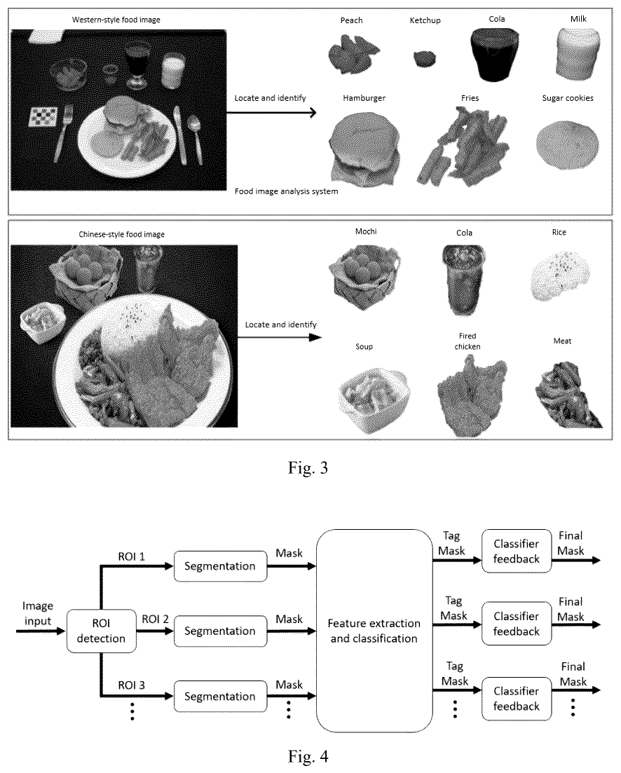

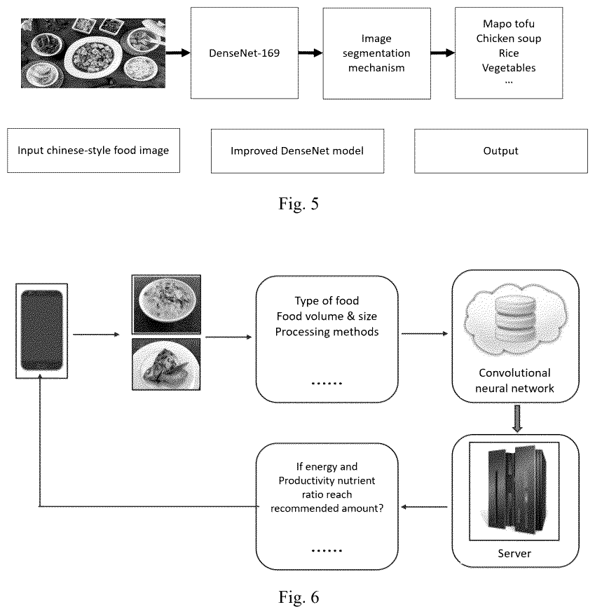

The present invention includes a food image segmentation process using image recognition technology, described as follows:
Step S21: food image segmentation and calibration, and blurred image processing;
Step S22: as a multi-classification problem, multi-category image classification is performed after segmentation and calibration, and the food category in the food image is extracted; sampling is based on the combination of threshold segmentation, region segmentation, edge segmentation and specific theory segmentation;
Step S23: feature extraction and classification; establishing image recognition models for the input image information, analyzing and extracting image features, and then developing a classifier with deep learning models to classify and recognize image features based on the extracted features;
Step S24: determining whether the accuracy of the classifier has been improved; if yes, returning the multi-hypothesis image to further execute feature extraction and classification;
Step S25: if no, outputting the final result (the category of the food in the image). An important feature of the present invention is image segmentation model training and testing, as shown in FIG. 3, described as follows:
Step 301: in the proposed classification system, LG is a channel for extracting features based on the entire image, and LL is a channel for extracting features based on local image blocks. f′(.) is the training feature set, and f′(.) is the feature of the image;
Step 302: segmenting the image after inputting, extracting LG based on the features of the entire image, and extracting LL based on the features of local image blocks;
Step 303: using the decision-making algorithm of tensor feature fusion to merge the global features and local features to obtain the target frame, and then judging whether the accuracy of the segmentation is improved, if yes, re-segmenting, and if no, outputting.

An embodiment of a food image segmentation system of the present invention is shown in FIG. 4, described as follows:
Step 401: using the image segmentation model to identify various foods;
Step 402: food image segmentation and calibration, blurred image processing and interference processing; during the calibration process, two groups of food images with common parts are transformed and synthesized into the same food image;
Step 403: identifying the ingredients of the food in the image, and identifying the categories of various foods;
Step 404: determining the statistical indicators such as the volume and weight of different ingredients through the images, and verifying the model hypothesis; the specific process of these statistical indicators is as follows: due to the scalability of the images, it is difficult to directly determine these statistics based on the size, but the proportion (%) of the segmented food can be determined by the image, and the weight of the food is set (such as 100 g or entered manually); the volume is computed and inferred according to the ratio, and the density of the corresponding food is found, so as to infer the corresponding weight.

An important part of the invention is the coding of the food image segmentation system, described as follows.
Step 501: the segmentation method segments the image into individual masks by regions of interest (ROI) detection; inputting the food image (matrix), binarizing the image, and extracting 3 values of each pixel (red, green and blue) or 1 value (black or white);
Step 502: extracting global features and local features, extracting the outline type of the food image, detecting the outline but not establishing a hierarchical relationship; using an approximation method to process and save the outline information; for example, a matrix outline is saved with 4 points;
Step 503: passing to the classifier and getting feedback; passing the output mask to the classifier, letting the classifier learn and feedback the category label of the food image in the data set; the returned image of the final mask after food image segmentation is the mask which has the same size as the original food image, but with a boolean value per pixel indicating the presence or absence of the object.

The embodiment here proposes a dieting image segmentation modeling, which consists of the following steps:
Step 601: a convolutional neural network based on an image segmentation and focusing mechanism; making the network focus on key areas and improving the ability to extract distinguishable semantic features of images;
Step 602: introducing a weighting mechanism into the field of image recognition, and proposing a weighting mechanism based on food image pixel level—DenseNet; in DenseNet, each layer takes an additional input from all previous layers and passes the feature of that layer to all subsequent layers; food image DenseNet uses a cascade method that each layer is receiving prior information from the previous layer to improve the network's ability to extract distinguishable semantic features, thereby improving recognition accuracy;
Step 603: using the image segmentation mechanism to complete deep learning, outputting the ingredients of the food in the image, and identifying the categories of various foods; after the inference of the deep learning model of dieting image segmentation obtains the types of food, according to the standard volume and weight of the food (reference) or the weight manually input by the diner (new label) and the estimated volume of other items according to the geometric space, the ontology knowledge of various foods (such as density) is combined to estimate the corresponding weight; according to the calorie prediction method, the calorie prediction of the target food can be further performed; during this process, the angle of the image (top view, side view, etc.) affects the complexity of volume computation; at the same time, various food images can be collected, and the corresponding food contained in each image can be manually marked, which includes category labels, volume, quality records, and specific calibration references; in terms of reference, the size of standard bowls and plates can also be used as a reference to extract the outline and volume of food.

The embodiment here proposes an image recognition system process, which includes the following steps:
Step 701: transforming food into images through mobile phone cameras, and then obtaining data such as food type, volume, weight and processing method through the data model, and putting the obtained data into the learning model to further optimize the algorithm;
Step 702: associating the server with other data sets to obtain whether the ratio of energy and production nutrients are in an appropriate range, and finally feedbacking the analyzed data and corresponding dietary suggestions to the user;
Step 703: image segmentation; according to the ratio of the pixels of each food in the image to the pixels of all foods, the proportion of the food in the whole package is obtained; by associating with relevant databases (such as the food composition table), the nutritional components contained in each food are obtained, and then the sum of the products of the ratio in the food package is obtained to obtain the content of each ingredient per 100 g of the food package; the total intake of each food is obtained according to the product of the total weight m of the food taken by the user, and the total nutritional component is obtained through computation;
Step 704: with real food data and algorithm optimization, the accuracy of food classification and volume/quantity estimation reach or exceed 75%;
Step 705: the specific calorie-nutrient component estimation method flow is as follows: first, images of different specifications [Top View or Side View] of the same type of food are used as input (Image Acquisition), and each image contains the calibration object and positioning for estimating the image scale factor; through the object detection network of the deep learning network to detect the food (Object Detection) and segment the target (Image Segmentation); the volume of each food is derived (Volume Estimation) through a specific food segmentation algorithm and reference standards; finally, the calorie of this type of food and the proportion of various ingredients ingested (%) and weight is estimated (Calorie Estimation) according to the density of this type of food.

In the embodiments of the present invention, the present invention also includes a computer-readable storage medium, on which a computer program is stored, and when the program is executed by a processor, the proposed method for personalized nutrition management with food image recognition models using deep learning can be realized.

Those of ordinary skill in the art can understand that all or part of the steps for implementing the above method embodiments can be completed by hardware related to computer programs. The aforementioned computer program can be stored in a computer-readable storage medium. When the program is executed, it executes the steps including the above-mentioned method embodiments; and the aforementioned storage medium includes ROM, RAM, magnetic disk or optical disk, and other various media that can store program codes.

A device for personalized nutrition management with food image recognition models using deep learning, wherein it includes a processor and a memory; the memory is used to store computer programs; the processor is connected to the memory for executing the computer program stored in the memory, so that the device for personalized nutrition management with food image recognition models using deep learning executes any one of the above method for personalized nutrition management with food image recognition models using deep learning.

Specifically, the memory includes various media capable of storing program codes such as ROM, RAM, magnetic disk, flash drive, memory card, or optical disk.

Preferably, the processor may be a general processor including a central processing unit (referred to as CPU), a network processor (referred to as NP); it may also be a digital signal processor (referred to as DSP), Application Specific Integrated Circuit (referred to as ASIC), Field Programmable Gate Array (referred to as FPGA), or programmable logic devices, discrete gate or transistor logic devices, and discrete hardware components.

The above descriptions are only examples of the invention, and are not used to limit the protection scope of the invention. For those skilled in the art, the application can have various modifications and changes. Any modification, equivalent replacement and improvement. made within the core content and principle of this invention shall be included in the protection scope of this invention.

1. A method for personalized nutrition management with food image recognition models using deep learning, wherein the method comprises: a user side obtains an food image to be taken by a user, and the food image is input into a trained food image recognition models using deep learning to obtain different types of food sub-images; computing the amount of nutrients contained in the food sub-images, and accumulating the nutrients in all the food to obtain the total nutrients intake of the user; setting intake thresholds of various nutrients, and comparing the total intake of various nutrients with corresponding nutrient intake thresholds to obtain a comparison result; according to the comparison result, type and quantity of taken food are adjusted, and nutrition management is completed;
wherein the process of training the food image recognition models using deep learning comprises:
Step 1: obtaining a food image dataset which contains different food images;
Step 2: pre-processing the data in the food image dataset, and dividing the pre-processed food images to obtain a training set and a testing set;
Step 3: using the object region detection algorithm to segment the food images in the training set into individual masks;
Step 4: performing feature extraction on each mask, obtaining global features and local features of each mask, and performing individual feature channel classification on each feature;
Step 5: using a decision-making algorithm for tensor feature fusion to merge the global features and local features to obtain the target frame;
Step 6: segmenting the food image according to the target frame; separating the pixel areas of different categories and different foods in the food image to complete general segmentation of the food image;
Step 7: identifying whether the types of each food image are the same, if yes, classifying semantic of each area and mark the category of each food image; if not, taking the food image as a new input, and returning to Step 4;
Step 8: numbering each food image based on semantic segmentation, outputting the image set.

2. (canceled)

3. The method for personalized nutrition management with food image recognition models using deep learning according to claim 1, wherein the pre-processing of the data in the food image dataset includes deduplication, image completion and image enhancement.

4. The method for personalized nutrition management with food image recognition models using deep learning according to claim 1, wherein the process of segmenting the food images in the training set into individual masks by the object region detection algorithm includes:
Step 1: binarizing the food image to obtain a binarized image; extracting 3 channel values or 1 channel value of each pixel in the binarized image;
Step 2: extracting contour type of the food image, and saving contour information by using approximation method; each element in the contour information saves a set of point set vectors composed of continuous food image points, and each set of food image point set represents a contour which is used as a feature for food image classification;
Step 3: segmenting the food image according to the contour information; after segmenting, returned image is the mask.

5. The method for personalized nutrition management with food image recognition models using deep learning according to claim 1, wherein the process of performing individual feature channel classification to the global features and local features of each mask comprises:
Step 1: performing affine transformation and feature extraction on the global information of each food image to obtain global features;
Step 2: performing feature extraction from each area in the food image, and fusing the local features of each area; the feature extraction methods include slices, segmented food information, and grids;
Step 3: using a deep learning network to classify individual feature channels.

6. The method for personalized nutrition management with food image recognition models using deep learning according to claim 1, wherein the decision-making algorithm of tensor feature fusion is used to merge the global features and local features, which includes:
Step 1: pre-processing the food image data, which includes subtracting the feature mean from each feature value, so that each feature has the same zero mean and variance; using tensors to construct the data structure of the 3 channels of the food image;
Step 2: computing covariance matrix of the tensor data, finding eigenvalues of the covariance matrix, arranging them from large to small, and selecting first k eigenvalues as number of features after dimensionality reduction;
Step 3: extracting the eigenvectors corresponding to the first k eigenvalues of the tensor data, so as to convert high-dimensional feature tensor into a k-dimensional eigenvector, and the k-dimensional eigenvector is the feature vector after dimensionality reduction fusion.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)
